(19) United States
(12) Patent Application Publication
(10) Pub. No.: US 2023/0320624 A1
(43) Pub. Date: Oct. 12, 2023

(54) DEVICE FOR COLLECTING EXHALED BREATH

(71) Applicant: SPECTRA 2000 S.R.L., Roma (IT)

(72) Inventor: Andrea MANNI, Roma (IT)

(73) Assignee: SPECTRA 2000 S.R.L., Roma (IT)

(21) Appl. No.: 18/037,881

(22) PCT Filed: Nov. 22, 2021
(86) PCT No.: PCT/IB2021/060810

§ 371 Date: May 19, 2023

(30) Foreign Application Priority Data
Nov. 23, 2020 (IT) 102020000028022

(51) Int. Cl. A61B 5/097

(52) U.S. Cl. CPC A61B 5/097; A61B 2010/0087

(57) ABSTRACT

A device for collecting exhaled breath allows a substantial air washing of the whole device which allows its re-use, after replacing the disposable portions. The device includes: a collecting terminal, having a mouthpiece grip which receives the exhaled breath; at least one collecting hole, for collecting a portion of exhaled breath from the collecting terminal, to be subjected to examination; least one discharge vent; and an aspirator device, which is connected to the at least one discharge vent of the collecting terminal through a respective discharge duct, and which includes at least one nanometric filter through which the exhaled breath sucked through is purified.

### FIELD OF THE INVENTION

The present invention relates to a device for collecting exhaled breath, i.e. the air exhaled by a user during their own respiratory act which is collected in a certain amount to be able to perform thereon a chemical analysis, for example within a procedure commonly known as Breath Test.

### BACKGROUND

The analysis of the exhaled breath, generally, is a method allowing to obtain information related to the state of health of one person. In fact, the air which is exhaled, that is released by the lungs during normal breathing, can be collected in a non-invasive way and in a substantially unlimited way, without altering the state of the user under analysis.

The Volatile Organic Compounds (VOCs), which are present in the exhaled breath, can be useful to identify bio-markers specific for some pathologies, such as for example lung cancer, asthma, pulmonary diseases and so on. Moreover, from the concentrations of VOCs in the exhaled breath it is possible to go back to their concentrations in the blood through known mathematic models.

Moreover, in gastroenterological field, the breath test allows to detect alterations of the gastroenteric system as bacterial contaminations (Helicobacter Pylori), intestinal malabsorption and alteration of the intestinal tract, which can appear by means of gastrointestinal disorders characterized by flatulence, meteorism, diarrhea, distension and abdominal cramps.

In these cases, the test consists in a collection of samples of exhaled air, before and after the ingestion of a specific sugar (glucose, lactose, fructose, lactulose) dissolved in water, thereto the user could be intolerant; the test aims at verifying the amount of hydrogen, methane and carbon dioxide in the exhaled breath.

Another examination of gastroenterological nature is the so-called Urea test, a quick diagnostic procedure to identify infections from Helicobacter pylori, a bacterium involved in gastritis, gastric ulcers and so on. The test relates to the capability of this bacterium to convert Urea into ammonia and carbon dioxide.

Other common tests involving the collection of the exhaled breath are spirometry, therewith the function of lungs is measured, particularly the volume and/or speed therewith the air can be inhaled or exhaled by a subject, and Alcohol Test, therewith, through the analysis of the exhaled air, the amount of ethanol in the blood is measured.

Spirometry is the most common and widespread examination of the respiratory function, which is performed with the help of an instrument for collecting exhaled breath called spirometer having a mouthpiece wherein the user has to insufflate for measuring the function of the lungs, particularly the volume and/or speed therewith the air can be inhaled or exhaled by a subject. The survey result consists of a series of values showing lung capability and volumes, apart from the degree of patency of the bronchi.

The alcohol test, instead, is performed with a device for collecting exhaled breath known as breathalyzer which performs a collection of the exhaled breath to detect the presence of ingested ethyl alcohol, by exploiting the fact that a small portion of alcohol, proportional to the ingested amount and then present in the blood, is removed through the alveolar air.

Similar tests can further be performed to find other medicinal substances or dopants present in the blood and transferred therefrom to the exhaled breath in the pulmonary alveoli.

By using one of the above-mentioned apparatuses, all falling in the common definition of device for collecting exhaled breath, the test execution is simple and not invasive, and then is safe for the user, but it can have risks deriving from the possible presence of pathogen agents in the exhaled breath, which can involve a risk for the operator, or for the subsequent user of the device for collecting exhaled breath, even when, as it happens in the common practice, the mouthpiece collecting the exhaled breath is disposable.

The US patent application Nr. 2017/119,280 A1 describes a device for the collection of the exhaled breath wherein, between the mouthpiece, which is useful for the user to exhale his/her own breath, and in the collecting terminal there is a filter useful to block viral or bacterial pathogen agents. However, this expedient forces the user to insufflate in a particularly vigorous way to overcome the filter resistance, thus causing a discomfort for the user, and provoking an alteration of the usual respiratory act.

Then, the device described in the art not only makes difficult the collection of the exhaled breath, until excluding categories of patients having reduced respiratory capabilities, but it leaves unsolved the contagiousness of the device portions which receive the exhaled breath downstream of the filter, and which then have to be of disposable type, including the same filter.

### SUMMARY

The solution idea of the above-outlined technical problem consists in performing an air washing of the exhaled breath not collected for the analysis, and in filtering in another section of the device the air volume used for such washing, and thus even the exceeding exhaled breath.

The technical problem underlying the present invention is to provide a device for collecting exhaled breath allowing to obviate the drawback mentioned with reference to the known art.

Such problem is solved by a device for collecting exhaled breath as defined in the description set forth below. It generally comprises:
a collecting terminal, which comprises a mouthpiece which receives the exhaled breath, a possible collecting hole, for collecting a portion of exhaled breath to be subjected to examination from the collecting terminal, and at least one discharge vent; and
an aspirator device, which is connected to said at least one discharge vent of the collecting terminal through a respective discharge duct, and which comprises at least one nanometric filter therethrough the exhaled breath sucked through said duct is purified.

The main advantage of the device for collecting exhaled breath according to the present invention lies in the fact of allowing the collection of the amount of the exhaled breath exhaled by the user required to perform the provided test, by evacuating subsequently the exceeding exhaled breath, performing a substantial air washing of the whole device, which allows its re-use, after replacing the disposable portions.

Advantageously, the suction of the exhaled breath from said collecting terminal takes place with a suction direction which is transversal to the direction wherein the exhaled breath is blown in the collecting terminal itself, by optimizing the effectiveness of the so-performed air washing.

### BRIEF DESCRIPTION OF THE DRAWINGS

The present invention will be described hereinafter according to a preferred embodiment example, provided by way of example and not with limitative purposes with reference to the enclosed drawings wherein:

FIGS. 1A, 1B and 1C show a side view, a front view and a top plan view, respectively, illustrating schematically an embodiment example of device for collecting exhaled breath according to the invention;

FIG. 2 shows a perspective view of a first terminal of device for collecting exhaled breath in glass tubes;

FIG. 3 shows a perspective view of a second terminal of device for collecting exhaled breath to perform a Urea test;

FIG. 4 shows a perspective view of a third terminal of device for collecting exhaled breath by means of a syringe;

FIG. 5 shows a perspective view of a spirometer used as terminal of device for collecting exhaled breath; and

FIGS. 6A, 6B and 6C show a sequence of perspective views of some components constituting a device used to collect and analyze the exhaled breath, by illustrating the different collection and washing phases.

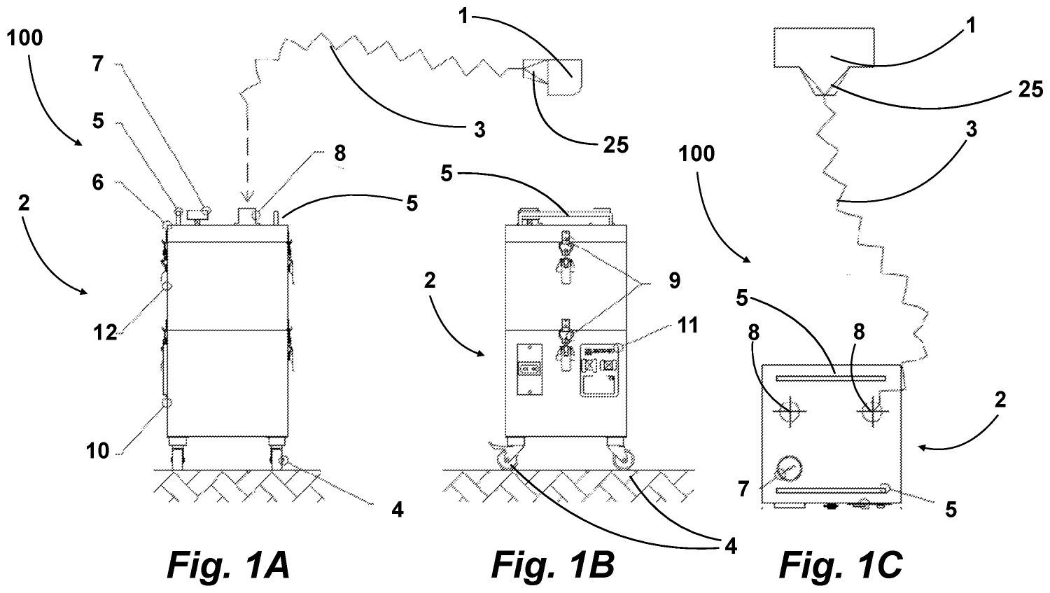

### DETAILED DESCRIPTION OF THE PREFERRED EMBODIMENTS

With reference to the figures, a device for collecting exhaled breath is designated as a whole with 100; it comprises one or more collecting terminals 1 of the exhaled breath, and a suction apparatus which is contained in a removable service module 2, in case provided with wheels 4.

The service module 2 comprises a handle 5 that eases the shifting thereof within consulting rooms or hospital environments.

At the top of the service module 2, the suction apparatus comprises a service plane 6 having controls and control devices, such as for example a display provided with keyboard and/or of touchscreen type and a manometer 7. Moreover, in the suction apparatus, one or more suction orifices 8 are provided, preferably equipped with a respective quick-type coupling, thereto it is possible to connect a tube, particularly a flexible hose, for example a corrugated tube (figures from 2 and subsequent ones), acting as discharge duct and which is designated with 3.

The suction orifices 8 are also provided with valves which can be actuated to open or close the respective suction orifice, particularly in case a discharge duct 3 is not connected thereto.

In this way, it is possible to use the service module for several collecting terminals 1, but it is also possible to maximize the washing of a reduced number of collecting terminals with respect to the number of the suction orifices.

On the side walls, the suction apparatus comprises quick closures 9 to access the internal compartment of the service module 2.

The suction apparatus comprises an aspirator device 10, arranged with at least one suction valve, an electric motor for the actuation of the suction device and a collecting container, provided at the base of the service module 2, thereon a control panel 11 is also provided.

At the upper portion of the service module 2, the suction apparatus instead comprises at least one nanometric filter, designated as a whole with 12, which in particular can be of the type which comprises a plurality of (not shown) interchangeable filtering barriers which can be replaced in the air path within the service module, from the suction orifices 8 on.

The object of the nanometric filter is to capture and block each liquid and solid element associated to the exhaled breath, and each possible, both viral and bacterial, pathogen agent.

Most pathogen agents have a size comprised between 0.05 and 1 μm; in particular, SARS-CoV-2 virus has a diameter measuring about 0.090 μm. The filters called ULPA (Ultra Low Penetration Air), according to standard EN1822-2009 of European Union, are the most suitable ones to keep particulate matter starting from 0.050 μm.

HEPA filters belong to the category of the so-called absolute filters, thereto even ULPA filters belong. The term “absolute filter” is justified by the fact that HEPA and ULPA filters have a high filtration effectiveness and, in particular, HEPA filters have a filtration effectiveness comprised between 85% and 99.995% (H14), whereas ULPA filters have a filtration effectiveness between 99.9995% and 99.999995%.

This type of filters can consist of filtering pieces of paper made of microfiber assembled in several layers, separated by septa made of aluminum. The filtering pieces of paper made of microfiber have the purpose of blocking the polluting solid particles existing in the fluid current to be treated.

ULPA and HEPA filters succeed in keeping particulate matter having sizes even lower than 0.3 μm; in fact, this is due to the fact that a particle with sizes lower than 0.3 μm, that is the so-called Most Penetrating Particle Size (MPPS), the most difficult size of particulate matter to be kept in the filter, does not pass therethrough as it is subjected to Brownian motion, therefore the particles move in a random path and collide with the surrounding environment as far as, upon meeting the filter fibers, they remain trapped therein: this is the case of viral pathogen agents, which have smaller size, such as for example SARS-CoV-2 virus.

In particular, the filters classified U15 have, by definition, an effectiveness higher than 99.995%, by making them suitable to the air filtering in the sanitary structures, virtually by removing each possibility of infection related to the use of the instruments for breath test or spirometry.

The aspirator 2 is arranged to act both on the alveolar exhaled breath of the user and on the air outgoing from the instrument. In order to be able to perform an air washing of the whole collecting device, the suction flow is very oversized with respect to the generated real flows, and then it has to have a volumetric flow rate higher than 30 m3/h, more preferably higher than 50 m3/h, for example 200 m3/h, with respect to the flow rate of 3-4 dm3/h received on the average from the terminal 1 by the user.

Such flow is then conveyed on the nanometric filter 12, that is capable of blocking particles having sizes smaller than 1 μm which, in a preferred version of the device according to the invention, is a filter of ULPA U15 type, which is capable of blocking powders, small drops, aerosol, bacteria and viruses.

Once sucked, the air flow which comprises even exhaled breath exhaled by the user, can be stocked, or however disposed of even by dispersion in the environment.

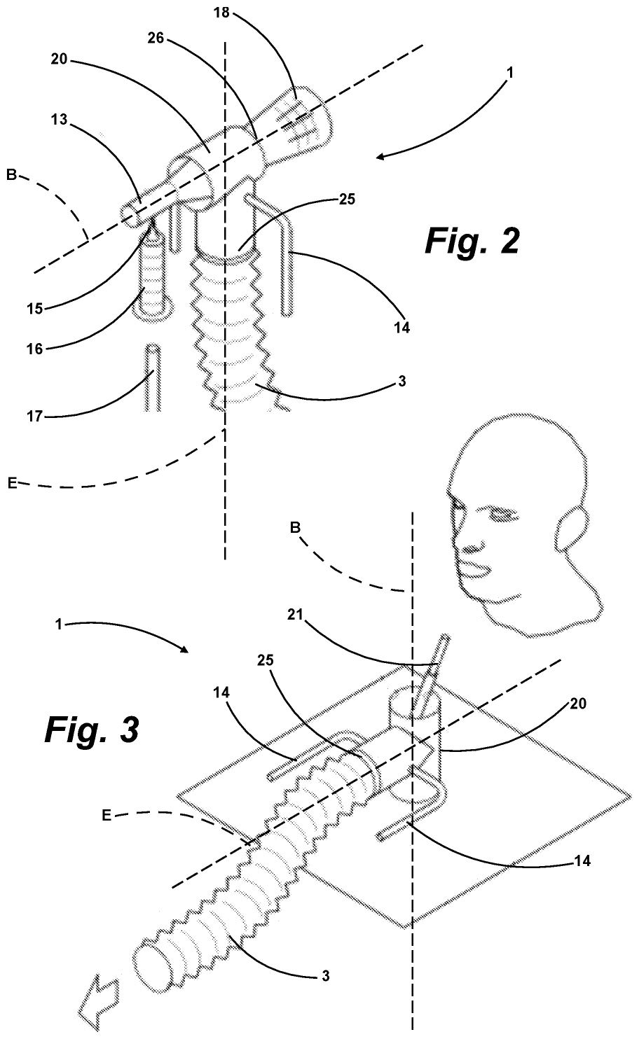

As a whole, with reference to FIGS. 2 to 6C, the collecting terminal 1 comprises a stiff casing 20 having a mouthpiece grip 13, which is provided with an appropriate non-return valve which is arranged to receive, through a (not shown) mouthpiece of disposable type which is connected to said mouthpiece grip 13, the exhaled breath insufflated or exhaled by a user.

The terminal 1 further comprises a grasping 14 which the user can use to approach the stiff casing 20 to his/her own mouth.

Conveniently, the grasping 14 consists of a pair of handles to allow even to a left-handed person to grasp the terminal correctly.

The stiff casing 20, forming the terminal 1, comprises at least one collecting hole 15, therefrom a portion of exhaled breath can be collected, to subject it subsequently to the examinations which have been requested. Even this collecting hole 15 can be conveniently provided with a non-return valve.

With reference to FIG. 2, on this regard the collecting hole 15 is connected to a first collecting device 16, which in the present example is of the type using adsorbent tubes 17 which represent one of the most used methods for collecting gases and vapors. These tubes typically have glass walls and include inside thereof different types of adsorbent substance (active carbon, silica gel, or porous organic polymers) which can be selected to trap only determined compounds. One of the advantages of the adsorbent tubes is to trap and store only the compounds of interest. Moreover, the chemical composition of the collected sample is not contaminated and through an easy desorption of the compounds it is possible to perform their analysis.

The terminal 1 comprises an expansion bag 18 connected to the stiff casing 20 by a suitable expansion vent 26; the expansion bag 18 works as collecting lung for the exhaled breath, allowing then the collection even in a higher amount than that provided by the stiff casing 20.

The position of the mouthpiece grip 13, the extension of the stiff casing 20 and the position of the possible collection bag 18 define a blowing direction of the user, identified by axis B in the figures.

The terminal 1 then, in the stiff casing 20, comprises a discharge vent 25 which is arranged to be connected to the discharge duct 3, which in the shown examples is a flexible hose.

The position of the discharge vent 25 with respect to the stiff casing defines a suction and washing direction of the exhaled breath from the terminal 1, identified by axis E in the figures.

This suction and washing direction of the exhaled breath from the terminal 1 is transversal to the blowing direction of the user, that is to the direction therewith the collecting terminal 1 develops; in particular, these two directions are perpendicular to one another.

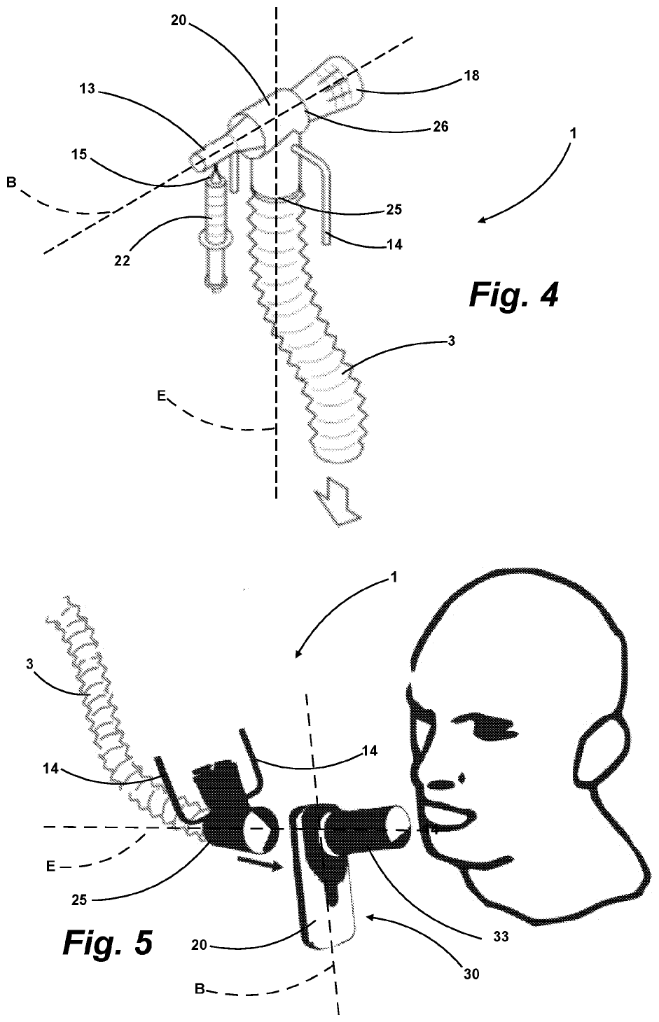

With reference to FIG. 4, the herein shown terminal 1 differs from that in FIG. 2 since it comprises a second collecting device 22 instead of the first collector, of syringe type.

Even in this example, in the stiff casing 20 the discharge vent 25 is provided, connected to the discharge duct 3 and the expansion vent 26 connected to an expansion bag 18.

Even in this example, the suction and washing direction of the exhaled breath from the terminal 1 is transversal to the blowing direction of the user, that is to the direction therewith the collecting terminal 1 develops; particularly, these two directions are perpendicular to one another.

With reference to the embodiment example shown in FIG. 3, a collecting device for performing the urea test has a collecting hole integrated in a straw 21 for collecting a small amount of exhaled breath, and in this case such straw 21 works as mouthpiece grip.

Since the distal end of the straw is open, it is inserted in a stiff casing 20 which defines a collecting direction B of the exceeding exhaled breath, and which is provided with a discharge vent 25, connected to the discharge duct 3, which defines a suction direction E transversal to the collecting direction B.

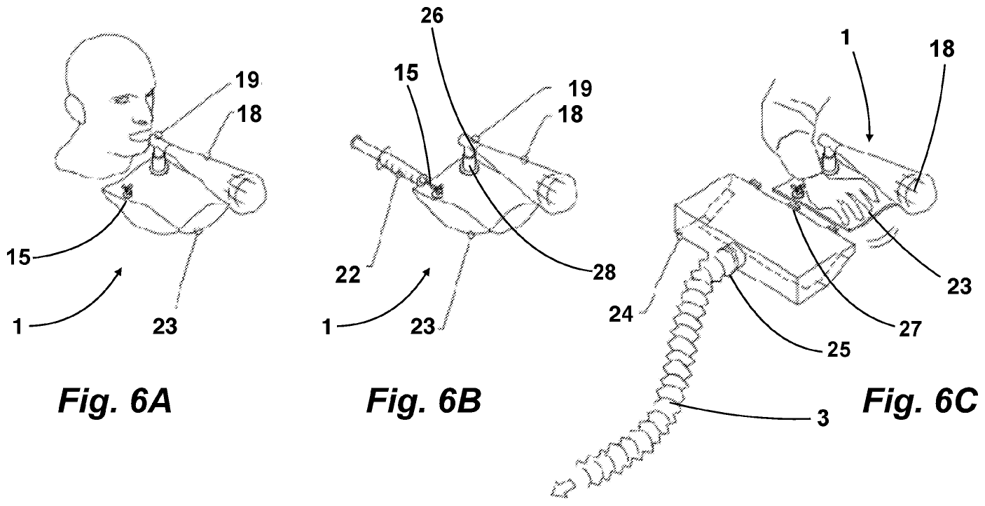

With reference to FIG. 6, a spirometer 30 is represented which includes a mouthpiece 33, working as mouthpiece grip, and a stiff casing 20 housing a device for measuring the flow rate of exhaled breath and its speed, in particular a digital turbine.

The casing 20 of the spirometer 30 has a longitudinal extension which defines a collecting direction B of the exhaled breath; on its rear wall it has a discharge vent 25, connected to the discharge duct 3, which defines a suction direction E transversal to the collecting direction B.

In the so far described examples, the stiff casing 20 forms a stiff portion of the terminal 1 wherein the discharge vent 25 and expansion vent 26, as well as the collecting hole 15 and the mouthpiece grip 13 are formed.

With reference to FIG. 6A, a collecting terminal 1 is represented comprising a flexible casing 23 which comprises said collecting hole 15 and which receives the exhaled breath from a user with the modes described with reference to one of FIGS. 2 to 4, with a mouthpiece 19 of the simpler, tubular, type which constitutes itself a mouthpiece grip. It is to be noted that, in this embodiment example, an expansion bag 18 is also provided, connected to an expansion vent 26.

The mouthpiece 19 and the expansion vent 26 are connected to a tubular element 28 which constitutes the stiff portion of the collecting terminal.

Once the exhaled breath is collected in the flexible casing 23, the portion necessary to the analysis is collected with a collecting device of syringe type, designated with 22 (FIG. 6B).

Once the collection has ended, the whole terminal 1, thus comprising the flexible casing and the expansion bag 18, can be connected to an intermediate container or a hood 24, for example a stiff casing, which is provided with suitable evacuation outlets 27 which are facing towards the flexible casing 23 which is emptied through the collecting hole 15 or another opening.

The hood 24, in case of portable type, comprises a stiff casing and, in turn, comprises a discharge vent 25 which is connected to the discharge duct 3 (FIG. 6C).

The hood 24 of this example then constitutes an additional stiff portion of the collecting terminal 1 which comprises the discharge vent 25.

In all above-described examples of collecting terminal 1, the discharge vent 25 and the discharge duct 3 are connected to a respective suction orifice of the suction apparatus of the collecting device 100.

Generally, once the exhaled breath has been exhaled by the user and then collected through the collecting hole 15, this mouth as well as the mouthpiece grip 13 remain substantially capable to be crossed by an air flow being sucked which is controlled by the exhalation apparatus, which is actuated subsequently to the collection of exhaled breath required to the requested examination.

In this way, the whole terminal 1 and the duct 3 are subjected to a powerful air washing which, subsequently, is addressed through the nanometric filters 12 which purify them, by trapping possible pathogen agents and even the liquid and aeriform residues transporting them.

Once the washing has been completed, performed by an automatic cycle set by an operator, the terminal 1 of the collecting device 100 can be used again; then it is ready to be connected to a new mouthpiece for a new collection.

It is also to be noted that the suction direction E, transversal and preferably perpendicular to the direction in which the exhaled breath is insufflated, in particular in tests with open configuration, such as Urea test or spirometry, is in a position so as not to alter in any way the result of the test itself.

To the above-described device for collecting exhaled breath a person skilled in the art, with the purpose of satisfying additional and contingent needs, could introduce several additional modifications and variants, however all within the protective scope of the present invention, as defined by the enclosed claims.

1. A device for collecting exhaled breath, comprising:
a collecting terminal, comprising a mouthpiece grip receiving the exhaled breath to be subjected to examination, and at least one discharge vent; and
a suction device, which is connected to said at least one discharge vent of the collecting terminal through a respective discharge duct and comprising at least one nanometric filter through which the exhaled breath sucked through said duct is purified.

2. The device for collecting exhaled breath according to claim 1, wherein the collecting terminal comprises at least one collecting hole, for collecting a portion of exhaled breath from the collecting terminal.

3. The device for collecting exhaled breath according to claim 1, wherein the collecting terminal comprises a stiff casing having said mouthpiece grip and said at least one discharge vent which define a blowing direction, whereas said at least one discharge vent defines a suction and washing direction of the exhaled breath from the collecting terminal which is transversal to said blowing direction.

4. The device for collecting exhaled breath according to claim 3, wherein the blowing direction and the suction and washing direction of the exhaled breath are perpendicular to one another.

5. The device for collecting exhaled breath according to claim 3, wherein the collecting terminal further comprises a handhold, usable to approach the stiff casing to a user's own mouth consisting of a pair of symmetrical bars projecting from said at least one discharge vent, thereby allowing a left-handed person to correctly grasp the collecting terminal.

6. The device for collecting exhaled breath according to claim 1, wherein said duct is a flexible hose.

7. The device for collecting exhaled breath according to claim 1, further comprising a removable service module, comprising one or more suction orifices thereto respective discharge ducts are connected by means of a corresponding quick coupling, inside thereof the aspirator and the nanometric filter are received.

8. The device for collecting exhaled breath according to claim 7, wherein said suction orifices are provided with respective valves which can be actuated to open or to close the suction orifice, when it is not connected to a discharge duct.

9. The device for collecting exhaled breath according to claim 1, wherein the aspirator implements, in each single discharge duct, a suction flow with a flow rate not lower than 30 m³/h.

10. The device for collecting exhaled breath according to claim 1, wherein the nanometric filter is a ULPA U15 type or higher.

11. The device for collecting exhaled breath according to claim 1, wherein said discharge vent is implemented in a rigid portion of the collecting terminal.

12. The device for collecting exhaled breath according to claim 11, wherein said rigid portion comprises an expansion vent connected to an expansion bag.

13. The device for collecting exhaled breath according to claim 1, wherein at least one collecting hole is connected to a respective collecting device.
